(19) United States
(12) Patent Application Publication
(10) Pub. No.: US 2008/0058840 A1
(43) Pub. Date: Mar. 6, 2008

(54) IMPLANTABLE COIL FOR INSERTION INTO A HOLLOW BODY ORGAN

(76) Inventors: Thomas E. Albrecht, Cincinnati, OH (US); Mark S. Ortiz, Milford, OH (US); David N. Plescia, Cincinnati, OH (US); Michael J. Stokes, Cincinnati, OH (US); Katherine L. Youmans, Cincinnati, OH (US); James W. Voegele, Cincinnati, OH (US); Mark S. Zeiner, Mason, OH (US); Jason L. Harris, Mason, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/469,564

(22) Filed: Sep. 1, 2006

(51) Int. Cl. A61B 17/08

(52) U.S. Cl. 606/153

(57) ABSTRACT

An implant for placement within a hollow body organ. The implant includes a member having an undeployed shape for delivery to the hollow body and a deployed shape for implantation therein. The member has sufficient rigidity in its deployed shape to exert an outward force against an interior of the hollow body so as to bring together two substantially opposing surfaces of the hollow body.

### FIELD OF THE INVENTION

The present invention has application in conventional open, laparscopic and endoscopic surgical instrumentation and methods as well application in robotic-assisted surgery. The present invention has even further relation to devices implanted within the stomach to induce weight loss.

### BACKGROUND OF THE INVENTION

Morbid obesity is a serious medical condition. In fact, morbid obesity has become highly pervasive in the United States, as well as other countries, and the trend appears to be heading in a negative direction. Complications associated with morbid obesity include hypertension, diabetes, coronary artery disease, stroke, congestive heart failure, multiple orthopedic problems and pulmonary insufficiency with markedly decreased life expectancy. With this in mind, and as those skilled in the art will certainly appreciate, the monetary and physical costs associated with morbid obesity are substantial. In fact, it is estimated the costs relating to obesity are in excess of one hundred billion dollars in the United States alone.

A variety of surgical procedures have been developed to treat obesity. The most common currently performed procedure is Roux-en-Y gastric bypass (RYGB). This procedure is highly complex and is commonly utilized to treat people exhibiting morbid obesity. Other forms of bariatric surgery include Fobi pouch, bilio-pancreatic diversion, and gastroplasty or “stomach stapling”. In addition, implantable devices are known which limit the passage of food through the stomach and affect satiety.

In view of the highly invasive nature of many of these procedures, efforts have been made to develop less traumatic and less invasive procedures. Gastric-banding is one of these methods. Gastric-banding is a type of gastric reduction surgery attempting to limit food intake by reducing the size of the stomach. In contrast to RYGB and other stomach reduction procedures, gastric-banding does not require the alteration of the anatomy of the digestive tract in the duodenum or jejunum.

However, gastric bands still require invasive surgical techniques. Recently, many new approaches to the treatment of obesity have been described in the art aiming to reduce invasiveness while maintaining effectiveness. First, restrictive procedures aim to reduce the amount of food a person can eat at a given time. One approach is endoscopic gastric restriction, which aims to create a small restrictive pouch in the proximal stomach by fastening anterior and posterior walls of the stomach together, simulating a vertical gastroplasty. Another approach is to use Restrictive sleeves. These are stent like structures, which are placed in the proximal most portion of the stomach and provide a restrictive outlet, preventing patients from overeating. Yet another approach is to use space occupying devices which maintain a constant volume in the stomach, limiting the amount of food a person can ingest at a given time. In yet another approach, physicians use balloons which expand in the stomach. While easy to install and reversible, these devices have been plagued by migration, leading to obstruction. Because of this, they have to be removed within 6 months.

### SUMMARY OF THE INVENTION

An implant for placement within a hollow body organ. The implant includes a member having an undeployed shape for delivery to the hollow body and a deployed shape for implantation therein. The member has sufficient rigidity in its deployed shape to exert an outward force against an interior of the hollow body so as to bring together two substantially opposing surfaces of the hollow body.

### BRIEF DESCRIPTION OF THE DRAWINGS

FIG. 1: U-shaped embodiment of the current design.

FIG. 2: Shows a view highlighting an embodiment of the exterior peaks and valleys.

FIG. 3: Internal structure of the U-shaped embodiment by removing the outer material.

FIG. 4: Cross sectional view highlighting the stiffening members.

FIG. 5: An additional cross sectional view highlighting the stiffness adjusting mechanism.

FIG. 6: Illustrates a second assembled circular or elliptical shaped embodiment.

FIG. 7: Cross sectional view shows the assembly means for the elliptical or circular embodiment.

FIG. 8: A segmented coil design in its undeployed state.

FIG. 9: A segmented coil design in its deployed state.

FIG. 10: A segmented coil design in its undeployed state.

FIG. 11: A segmented coil design in its deployed state.

FIG. 12: A segmented coil design in its deployed state.

FIG. 13: Means for access to tensioning rods and wires.

FIG. 14: A two-segment device for creating closed loop coils in an undeployed state.

FIG. 15: A two-segment device for creating closed loop coils in a partially deployed state.

FIG. 16: A two-segment device for creating closed loop coils in its fully deployed state.

FIG. 17: Means for controlling coil shape through uniform interior geometrical design.

FIG. 18: A possible cross-section of the coil in FIG. 17.

FIG. 19: Another possible cross-section of the coil in FIG. 17.

FIG. 20: Means for controlling coil shape through non-uniform interior geometrical design.

FIG. 21: Possible basic embodiment of the current invention.

FIG. 22: Possible basic embodiment of the current invention.

FIG. 23: Possible basic embodiment of the current invention.

FIG. 24: Possible basic embodiment of the current invention.

FIG. 25: Possible basic embodiment of the current invention.

FIG. 26: Possible basic embodiment of the current invention.

FIG. 27: Possible basic embodiment of the current invention.

FIG. 28: Possible basic embodiment of the current invention.

FIG. 29: Possible basic embodiment of the current invention.

FIG. 30: Possible basic embodiment of the current invention.

FIG. 31: Possible basic embodiment of the current invention.

FIG. 32: Unaltered view of the stomach prior to device insertion.

FIG. 33: Unaltered cross-sectional view of the stomach prior to device insertion.

FIG. 34: Modified view of the stomach following device insertion showing stomach flattening.

FIG. 35: Altered cross-sectional view of the stomach following device insertion showing stomach extension in plane of device.

FIG. 36: Modified view of the stomach following device insertion showing stomach flattening.

FIG. 37: Altered cross-sectional view of the stomach following device insertion showing stomach extension in plane of device.

FIG. 38: Modified view of the stomach following device insertion showing stomach flattening.

FIG. 39: Altered cross-sectional view of the stomach following device insertion showing stomach extension in plane of device.

FIG. 40: Modified view of the stomach following device insertion showing stomach flattening.

FIG. 41: Altered cross-sectional view of the stomach following device insertion showing stomach extension in plane of device.

FIG. 42: Initial step to deploy an open looped embodiment of the current invention.

FIG. 43: Intermediate step to deploy an open looped embodiment of the current invention.

FIG. 44: Intermediate step to deploy an open looped embodiment of the current invention.

FIG. 45: Intermediate step to deploy an open looped embodiment of the current invention.

FIG. 46: Intermediate step to deploy an open looped embodiment of the current invention.

FIG. 47: Final step to deploy an open looped embodiment of the current invention.

FIG. 48: Initial step to deploy a closed loop embodiment of the current invention.

FIG. 49: Intermediate step to deploy a closed loop embodiment of the current invention.

FIG. 50: Intermediate step to deploy a closed loop embodiment of the current invention.

FIG. 51: Intermediate step to deploy a closed loop embodiment of the current invention.

FIG. 52: Intermediate step to deploy a closed loop embodiment of the current invention.

FIG. 53: Final step to deploy a closed loop embodiment of the current invention.

FIG. 54: Initial step to deploy a closed loop embodiment of the current invention.

FIG. 55: Intermediate step to deploy a closed loop embodiment of the current invention.

FIG. 56: Intermediate step to deploy a closed loop embodiment of the current invention.

FIG. 57: Intermediate step to deploy a closed loop embodiment of the current invention.

FIG. 58: Intermediate step to deploy a closed loop embodiment of the current invention.

FIG. 59: Final step to deploy a closed loop embodiment of the current invention.

FIG. 60: Initial step to retrieve an open looped embodiment of the current invention.

FIG. 61: Intermediate step to retrieve an open looped embodiment of the current invention.

FIG. 62: Intermediate step to retrieve an open looped embodiment of the current invention.

FIG. 63: Intermediate step to retrieve an open looped embodiment of the current invention.

FIG. 64: Intermediate step to retrieve an open looped embodiment of the current invention.

FIG. 65: Final step to retrieve an open looped embodiment of the current invention.

FIG. 66: Open looped embodiment of the current invention.

FIG. 67: Possible position within the stomach for an open looped device.

FIG. 68: Possible position within the stomach for an open looped device.

FIG. 69: Possible position within the stomach for an open looped device.

FIG. 70: Overlapping open looped embodiment of the current invention.

FIG. 71: Possible position within the stomach for an overlapping open looped device.

FIG. 72: Possible position within the stomach for an overlapping open looped device.

FIG. 73: Possible position within the stomach for an overlapping open looped device.

FIG. 74: Closed loop embodiment of the current invention.

FIG. 75: Possible position within the stomach for a closed loop device.

FIG. 76: Possible position within the stomach for a closed loop device.

FIG. 77: Possible position within the stomach for a closed loop device.

FIG. 78: Possible location and method to fixate or attach an open looped device within the stomach to prevent migration.

FIG. 79: Possible location and method to fixate or attach an open looped device within the stomach to prevent migration.

FIG. 80: Possible location and method to fixate or attach a closed loop device within the stomach to prevent migration.

FIG. 81: Possible location and method to fixate or attach a closed loop device within the stomach to prevent migration.

### DETAILED DESCRIPTION OF THE INVENTION

Below is described devices and methods for inducing weight loss in patients is designed to hold the stomach wall largely taut, reducing effective volume per tissue surface area and inducing a prolonged hormonal response within the body reducing the desire to eat and biasing stretch receptors to send an early sense of satiety. A semi-rigid closed loop (i.e. circular, elliptical, figure eight, etc) or open loop (i.e. U-shaped) implant in the stomach lumen is designed to apply an outward radial force in a single plane flattening the stomach. The plane in which the coil sets may change over time due to stomach motions. The preferred device can be easily installed and removed endoscopically and has design features that prevent migration and erosion. Although less desirable, laparoscopic, open surgical techniques or a combination may also be used to place such a device.

The device effectively flattens the gastric lumen, reducing effective volume per surface area of stomach tissue, increasing gastric wall tension and biasing stretch receptors to send an early sense of satiety. The stretching of the stomach tissue inhibits gastric motility and delays gastric emptying. In addition, the device can be configured to rest against and partially block the pyloric outlet, delaying gastric emptying. In addition, the device could provide a restrictive element just distal to the esophageal gastric junction. Literature supports the notion that a prolonged hormonal and/or neurological response may be released from the stomach, reducing the desire to eat. Optimal configurations may target specific regions of the stomach activating multiple combinations of these mechanisms.

The device is preferably inserted trans-orally while deformed, folded, or otherwise placed into a relatively straight position and subsequently takes its final shape while inside the stomach lumen. The final configuration is flexible enough to allow some compression and movement relative to the stomach mucosa, preventing erosion, but rigid enough to prevent unwanted buckling and proximal or distal migration. Erosion may be further minimized through optimal material selections, exterior shapes that vary pressures on the lumen walls, and exterior shapes that encourage movement of the device within the stomach so that no single location is always subject to the same contact loads from the device. Additionally the device could change its size periodically to relieve pressure on the gastric wall at certain times of the day.

Optimally, a single device may be used for all stomach sizes. This could require the device to be adjustable before, during, or after the procedure. Alternatively, the device could be designed to adjust automatically over time through the use of biodegradable materials or a constant force spring mechanism. To aid with sizing, placement and adjustment, the devices may be designed to be visualized through indirect means (e.g. radio-opaque, etc.). Alternatively, multiple coil sizes may be available with the proper size selected at the time of the procedure. Additionally, the length of the device can be modified prior to deployment by trimming the device and/or removing segments.

The device should be designed to be retrievable in the event of unforeseen complications. However, it may be desirable to retrieve the device after a predefined period of time, either to end the intervention, to make adjustments to the device, or to replace the device with different characteristics (size, shape, stiffness, features, etc.) Specific details of endoscopic device retrieval will be highly dependent on the concept selected, but many fall into two broad classes; devices that are pulled out the way they were inserted and devices that require manipulation or modifications prior to retrieval. Many open looped designs fall into the former. Features such as hooks, loops, and holes may be present and can be snared or grabbed allowing the device to be pulled out through a device similar or identical to the deployment means. Closed loop devices may more often require some form of manipulation to allow the device to be retrieved. Mechanical snaps, tensioning wires, or other means of holding the loop closed likely need to be released to allow endoscopic removal of the coil. Alternative methods exist and are specific to the coil design, but all retrieval methods should be atraumatic to the patient.

Devices may be designed so that retrieval is not expected. One option is that the coil is intended to be a permanent implant. Alternatively, however, the coil may be designed so that part or the entire device is bioabsorbable, digestable, or otherwise designed to degrade over a controlled time in a predefined manner. In the event only portions of the device are meant to degrade, non-degradable portions should be sized so that they can naturally pass from the body, or should be large enough so that they remain in place without impacting the patient until time for retrieval. Degradable sections may be designed to release medicines or other materials that benefit the patient. Although designed so retrieval is not necessary, all bioabsorbable concepts should be removable.

Finally, whether permanent, removable, or degradable, devices may perform optimally when seated in specific regions of the stomach. Specific coil shapes may encourage the coil to seat itself primarily in the targeted region. Alternatively, additional fixation means (suture, adhesives, additional features, etc) may be used, but should be reversible for easy removal for temporary applications. In addition, these fixation means could help prevent unwanted migration of the device.

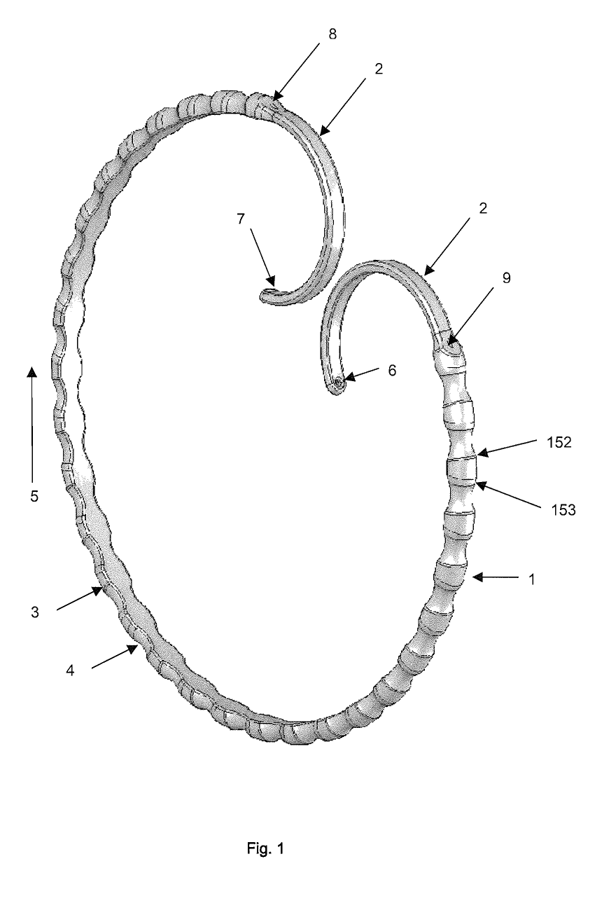

FIG. 1 illustrates the open loop U-shaped embodiment of the design. Section 1 of the device is semi-rigid, largely U-shaped and atraumatic portion that is designed to take a largely straight configuration during deployment and take the shown curved configuration once deployed in the lumen. It is designed to apply an outward force on the gastric lumen, keeping the tissue taut but allow enough flexibility to prevent damage to the stomach tissue.

Section 2 is one of two ends of the device that are coiled into a smaller diameter. These ends are large enough in diameter and stiff enough to prevent migration proximal or distal once placed in the gastric lumen. Also, they prevent damage of the stomach tissue by providing a blunt end.

The peaks 3 and valleys 4 along Section 1 of the device serve several purposes. They provide a path around which gastric content can travel through to the pylorus, preventing an obstruction while slowing gastric emptying. These features also prevent erosion of the device into the gastric tissue by providing a gripping surface for the stomach's peristaltic movements to propel the device in a circular motion 5. Alternating “sharper” (atraumatic) edges 152 and “smoother” edges 153 promote a preferential direction for the motion 5. As the peaks 3 and valleys 4 move from one portion of the stomach wall to the next, blood flow to the tissue is maintained. However tissue stresses in contact with the peaks 3 will be higher than tissues stresses in contact with the valleys 4. As the coil moves 5 within the stomach tissue, stresses in contact with the device vary continuously, minimizing trauma (leading to erosion, ulcers, etc.) to the mucosa. In addition to these features, the peaks 3 and valleys 4 provide a stimulating effect to the gastric mucosa tissue, sending an additional sense of satiety to the patient.

Holes 6 and 7 are used to aid in device removal by providing a feature to securely hold the device.

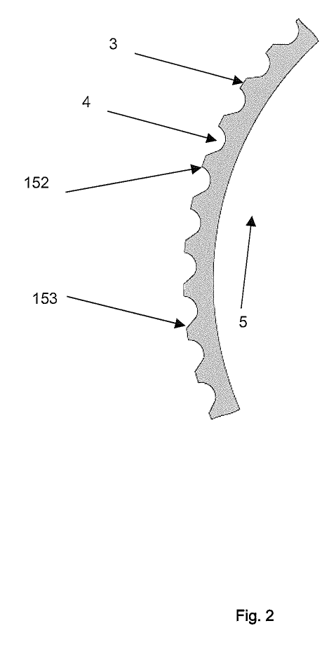

Holes 8 and 9 represent two ends of a lumen that travels the whole distance of the device. This lumen is designed to accept a stiffening element to allow for some degree of adjustability. FIG. 2 shows another embodiment highlighting the peaks 3 and valleys 4. Here “sharper” edges 152 and “smoother” edges 153 are created by adjusting the angles of the intersecting surfaces. As shown, the configuration of edges 152 and 153 promote a clockwise circular motion 5.

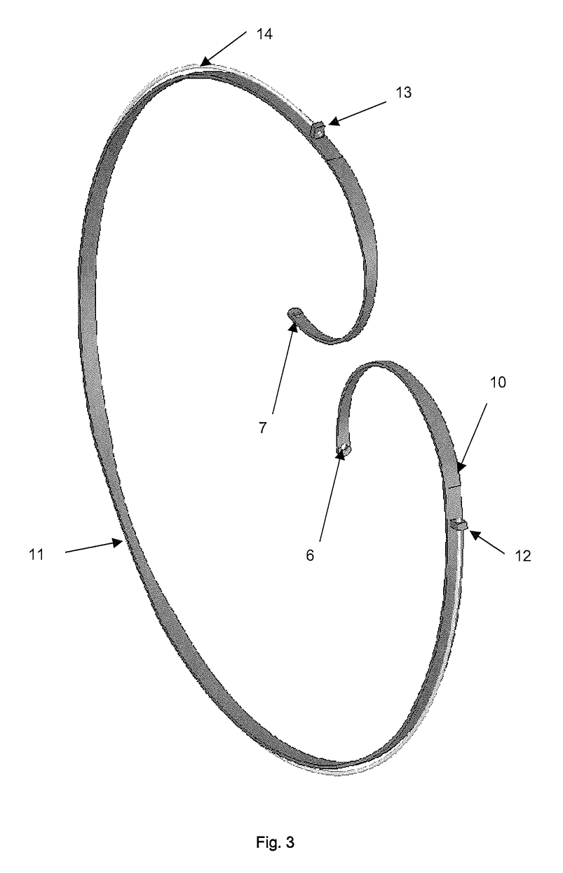

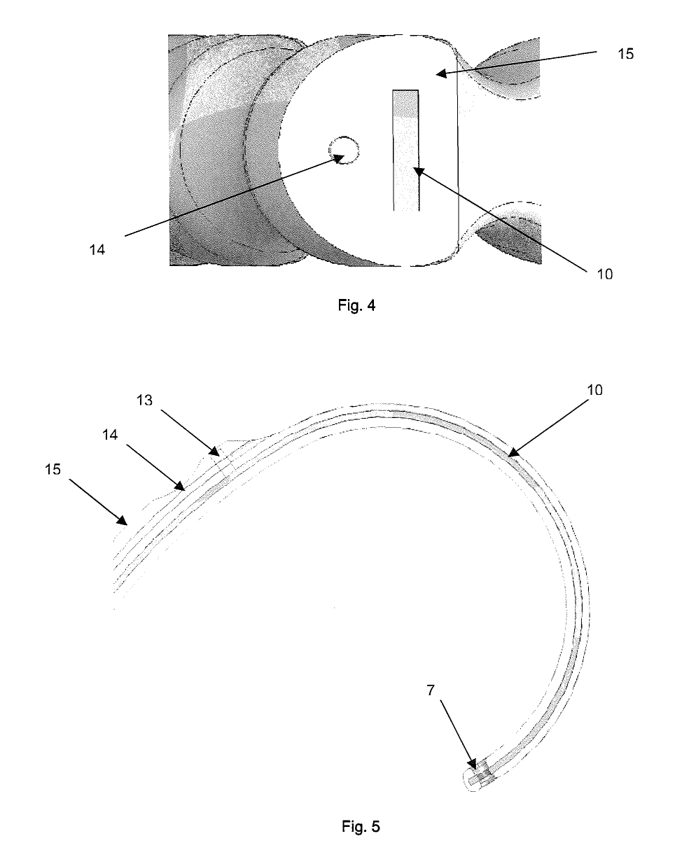

FIG. 3 shows the internal stiffening members of the design having removed the outer atraumatic material 15 (FIG. 4) Band 10 is a super elastic member such as nitinol or a shape memory material such as nitinol or a shape memory polymer that is designed to be largely straight during insertion but take the illustrated configuration once installed in the gastric lumen. The band 10 can be induced to conform to its shape-memory configuration through thermal, chemical, magneto, or optical means depending on the material selection. It provides the bulk of the outward forces required to stretch the gastric lumen and can be designed to be more or less stiff at certain points. Band 10 stiffness can be varied through changes in band geometry, outer material 15 (FIG. 4) geometry, material homogeneity, bioabsorbable degradation profiles, and material treatments (i.e. adjustments to temper) to name a few. In plane and out of plane stiffness can be altered by these means. In FIG. 3, a multitude of narrow sections 11 of the band are provided to allow for out of plane flexing during installation and during violent contraction of the stomach should they occur.

Adjustment element holders 12 and 13 connect adjustment element 14 to the band 10. The adjustment element is rigidly attached at one adjustment element holder 12 but adjustably attached at the second 13, allowing for modification of the shape and stiffness of portion 1 (FIG. 1). The adjustment element could be made of a metal wire. Alternatively, the adjustment element 14 could be made of shape memory nitinol. When activated (by thermal, chemical, magneto, optical, or other means), the element 14 would shorten, widening the device along portion 1 (FIG. 1). In addition, the element 14 could be simply replaced with a different stiffness element to adjust the shape along portion 1. In addition, the adjustment element could be removed and the lumen could be filled with a liquid epoxy that would subsequently harden and hold the device in its desired configuration. The epoxy could naturally cure or be cured through intervention (e.g. ultraviolet light). Finally, the adjustment element could be a single or numerous shape memory polymer elements that would shorten to a predetermined length after being exposed to a certain light frequency, allowing for controlled adjustment.

FIG. 4 shows a cross-sectional view of the device, highlighting the orientation of the band 10, the adjustment element 14 and the outer atraumatic material 15. This outer material 15 would be made of a flexible and biocompatible material that could be bioabsorbable or biodegradable. Candidate materials include but are not limited to silicone, polypropylene, PDS and vicryl. Additionally, it could elude drugs such as PPI's and/or anti-nausea medications.

FIG. 5 is an additional cross-sectional view showing the band 10, the adjustment element 14, the adjustment element holder 13, the removal hole 7 and the outer material 15.

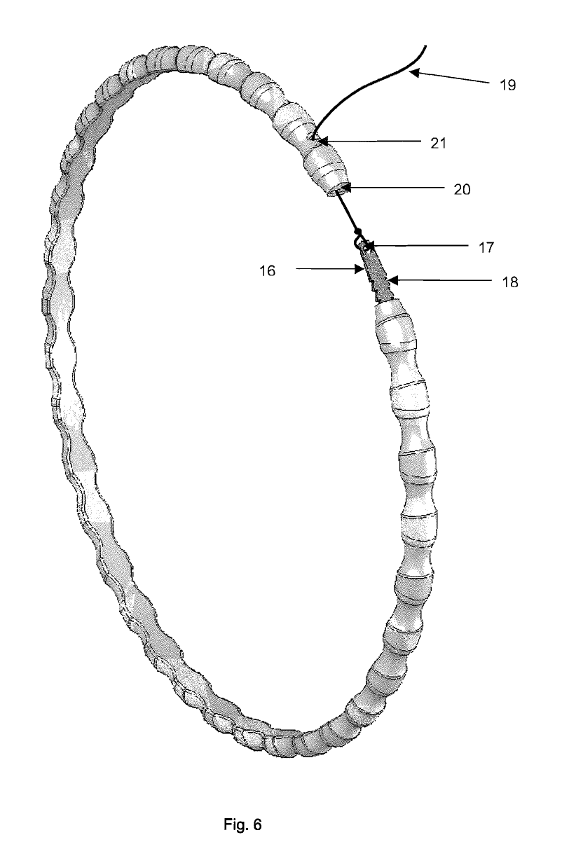

FIG. 6 shows an alternative embodiment of a closed loop device in which the device in its final configuration is a complete circle or largely elliptical shape by connecting both ends of the device in the gastric lumen. Lock tab 16, which could be part of or separate from band 10 in FIG. 3, has a pull wire hole 17 and one-way engagement notches 18. A flexible wire or string 19 is connected to hole 17 and run through slot 20 and out hole 21. Pulling on wire 19 will engage and lock tab 16 into slot 20, forming a complete semi-rigid circular or elliptical shape. To unlock the device for removal, the latching surfaces inside of slot 20 (not shown) would be moved out of the way to allow for the band tab 16 to be removed from slot 20. Alternatively the latching surface could be made of a shape memory polymer that when exposed to a certain frequency of light (or other activating medium), would move out of the way, clearing the path for the lock tab 16 to be removed from slot 20.

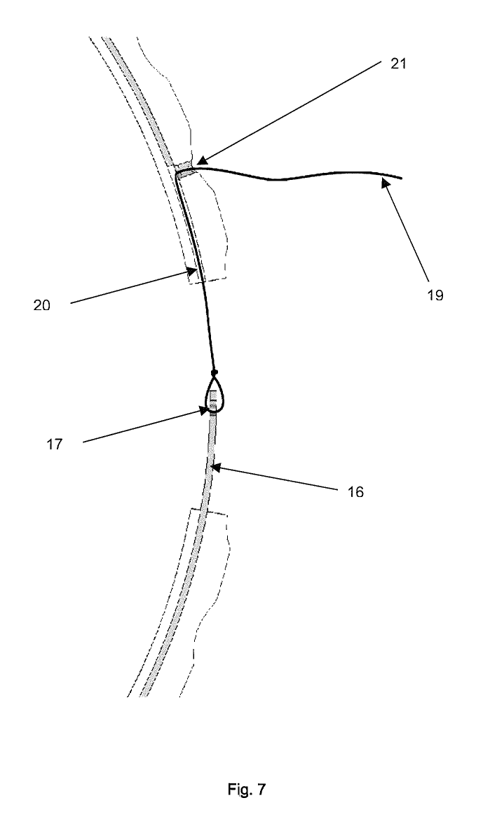

FIG. 7 is a cross-sectional view of the embodiment of FIG. 6, highlighting the elements just discussed.

FIG. 8 shows an undeployed 100 device created from multiple segments 104 (FIG. 9.) Segments 104 can be connected by methods such as snap fits and hinges or the segments 104 may contain a lumen through which a wire 105 (FIG. 9) is passed. The overall size and shape of the device 101 (FIG. 9) may be controlled by adding or removing segments 104 prior to delivery.

FIG. 9 shows a deployed 101 closed loop device resulting from the deployment of the device 100 in FIG. 8. The design of the segments 104 help define the final device shape 101.

Segments 104 may be relatively rigid or deformable. For deformable materials, segments of smaller diameter 106 (FIG. 11) may be used to achieve larger deformations for a given wire 105 tension. Other modifications to segment geometry (not shown) can achieve changes in shape at a given wire 105 tension including changes in length, changes in cross sectional area, and consistency in cross sectional area. Additionally, segments 104 may be partially or completely made of bioabsorbable materials so that the stiffness of the segments 104 will alter over time. Changes to segment stiffness can be used to increase or decrease the overall characteristics of the coil.

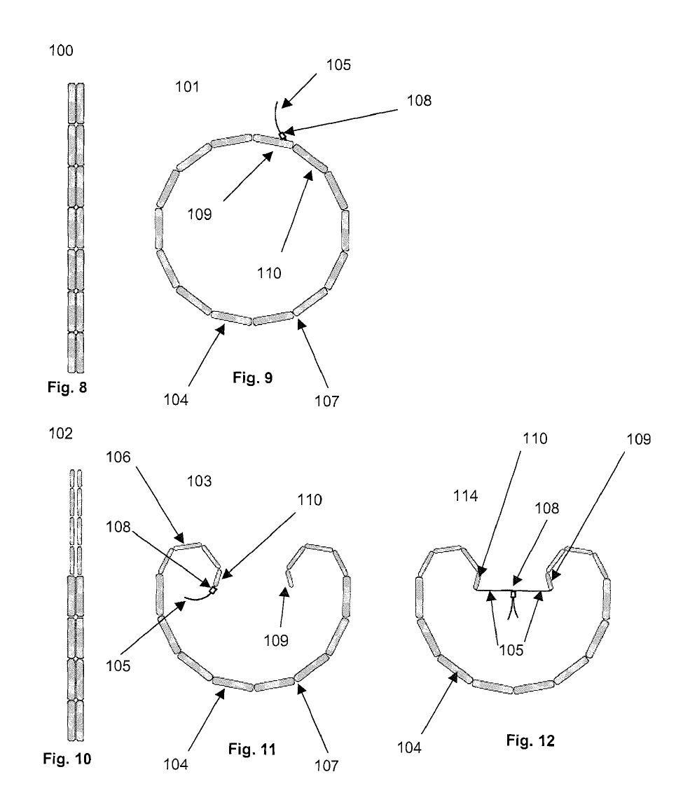

Another feature of the device that can help define the shape of device 101 (103 FIG. 11 and 114 FIG. 12) is the interface 107 between segments 104. Segment ends may be designed to mate in a unique orientation to adjacent segments. This mate may be held in place by the tension in the wire 105, (which could be designed to allow motion periodically while always returning to “home”) or the mate may be rigid and semi-permanent through the use of snap fits. Numerous methods for alignment are well known including mating mechanical features, magnetic alignments, etc. It may also be desirable for segments 104 to remain in contact at the interface 107, but have freedom for rotation and/or restricted motion. Wire 105 tension along with more specialized mechanical and magnetic mates can accomplish this intent.

Tension in the wire 105 can be preloaded into the device or applied after deployment. Physical properties of the wire 105 can limit options in some cases. If an extensible wire 105 is chosen, the segments 104 of the device may be separated, folded, and/or held in the desired shape (100 and 102 for instance). These motions increase the tension in the wire 105. When released, the increased tension can pull the segments 104 together so that the interfaces 107 mate together as designed (either into a unique orientation that could possibly snap together or at least come together so that the interfaces 107 are approximated). Final wire 105 tension ideally can be set prior to deployment, although adjustments to the tension may be made following deployment. A feature of a device deployed with an extensible wire 105 is the deployed device (e.g. 101, 103, and 114) can be more deformable and able to adapt to temporary changes in stomach geometry.

For an inextensible wire 105 the ability to stretch the wire prior to deployment is not present minimizing opportunities to allow the pretension to auto-locate the segments 104 without intervention. However, once deployed, manual tensioning can be used to auto-locate segments 104 so that interfaces 107 mate together as intended. A feature of a device deployed with an inextensible wire 105 is the deployed device (e.g. 101 and 103) can be less deformable and will prevent or minimize temporary changes in stomach geometry.

Methods to increase tension in deployed segmented devices (e.g. 101, 103, and 114) containing a wire 105 including but not limited to the use of knotting elements 108 (such as one-way ratchet locks.) These knotting elements 108 can be deployed over a single wire 105, or over two or more wires 105. In the case of a single wire 105, one end of the wire is attached to an end segment 109 of the coil. The wire 105 passes through all segments 104 and passes out of the opposing end segment 110.

For numerous coil designs, the knotting element can be passed over the wire down to end segment 110. The tension can be set as desired so that the resulting shape is obtained. Once properly adjusted, the knotting element 108 is deployed and left in place. For a closed loop coil 101 (FIG. 9), the wire 105 may pass out of end segment 110 and then back through end segment 109 creating interface 107. Alignment and mating can be performed similar to the embodiment in FIG. 6, or a knotting element 108 can be passed over a single wire 105 as described above.

FIG. 10 shows an undeployed device 102 created from multiple segments 104. The overall size and shape of the device 103 (FIG. 11) or device 114 (FIG. 12) may be controlled by adding or removing segments 104 prior to delivery. This embodiment may have some or all of the characteristics of the device described in FIGS. 8-9. This device (102, 103, and 114) is an open looped coil created from segments 104 of different shapes and properties.

FIG. 11 shows a deployed device 103 created from multiple segments 104 of different shapes and properties. This open looped device 103 differs from the device 114 in FIG. 12 through the way in which the tension in the wire 105 is set. This figure shows a wire 105 permanently attached to the segment at end 109, passing through all segments and out segment end 110. For the open looped coil 103, the knotting element 108 can be passed over the wire down to end segment 110. The tension can be set as desired so that the resulting shape is obtained. Once properly adjusted, the knotting element 108 is deployed and left in place.

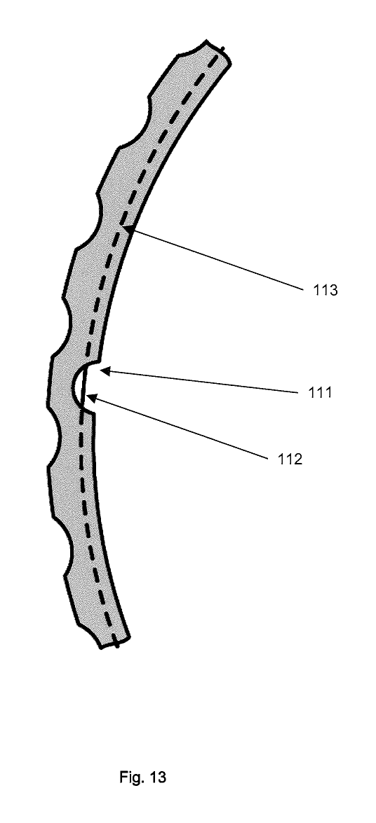

FIG. 12 shows a deployed device 114 created from multiple segments 104 of different shapes and properties. This open looped device 114 differs from the device 103 in FIG. 11 through the way in which the tension in the wire 105 is set. This figure shows a wire 105 that is not fixed to end segment 109 so that an excess of wire 105 extends from both end segments 109 and 110. In this case, knotting element 108 can be passed over both ends of wire 105 as shown in device 114. Tension applied to both ends will approximate, mate, and/or deform segments 104 creating deployed coil shapes 114. FIG. 13 shows how a notch 111 can be provided in Section 1 (FIG. 1) of the device to access a stiffening member 112 (or tensioning wire 105) that runs in channel 113. This access point can be used to cut the stiffening member to allow for easier removal of the device.

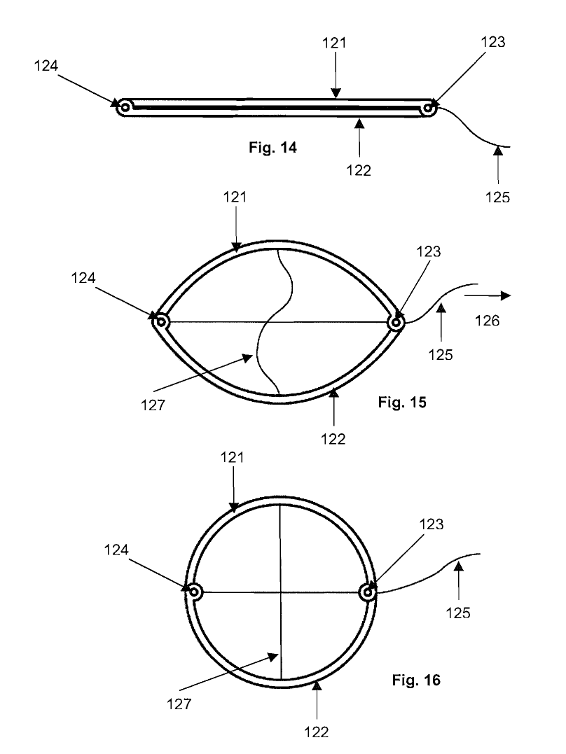

FIG. 14 shows an alternative embodiment of the current design in which two flexible members 121 and 122 whose ends are attached at pivot points 123 and 124. A tensioning wire 125 is adjustably attached to pivot point 123 and rigidly attached to pivot point 124.

FIG. 15 shows steps in the deployment of the embodiment of FIG. 14. As the tensioning wire 125 is pulled in direction 126, the device expands from its flat insertion configuration to its final deployed expanded position. An additional tension wire 127 can be provided to better hold the device in its final configuration when acted upon by the natural movements of the stomach lumen.

FIG. 16 shows the final, deployed configuration of the embodiment of FIG. 14. The length of members 121 and 122 and wires 125 and 127 can be selected or adjusted to define circular or elliptical deployed configurations.

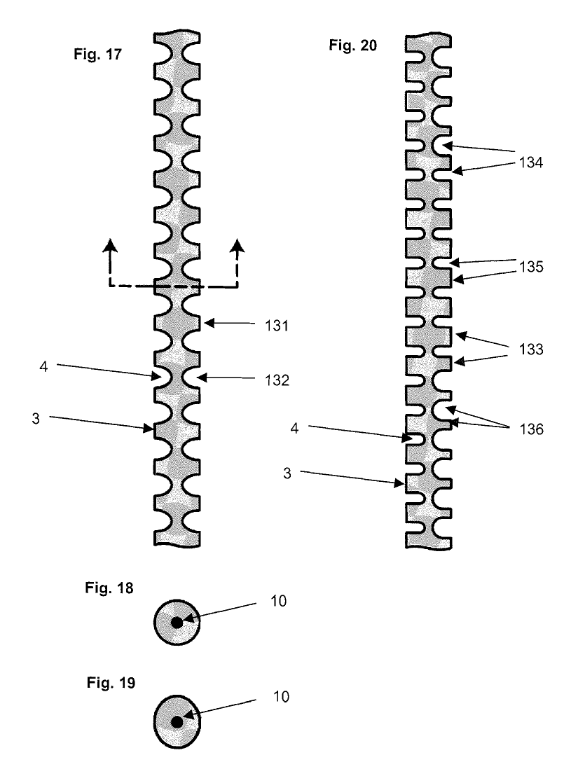

FIG. 17 shows a section of coil in an undeployed configuration. The coil may have a band 10 (FIG. 18, 19) to strengthen the coil or to bias the direction of bending of the device. The device can be designed so that regular shaped peaks 3 and valleys 4 exist on one side (designed for contact with the stomach wall). The interior side of the coil will also have peaks and valleys that are designed to come in contact with adjacent pieces when deployed. The interior peaks 131 and valleys 132 in this embodiment are of uniform shape and size. The size and shape of the peaks 131 and valleys 132 control the deformed shape of the coil. Wider peaks 131 and narrow valleys 132 will allow less bending whereas narrow peaks 131 and wide valleys 132 will allow greater amounts of bending. Changes in geometry (angle, radius, depth, width, height, etc) allow excellent control over deformed coil shapes. For instance, a coil with uniform peaks 131 and valleys 132 will likely deform (when fully engaged) into a coil of constant radius. Cross sectional areas of the coil may be round (FIG. 18), elliptical (FIG. 19), or of other smooth shapes. The coil may contain holes 8 and 9 for stiffening elements (not shown).

Coils of this embodiment can be designed to be very rigid in one plane, but deformable within the plane allowing the coil to adjust to its surroundings. Further, the shape of the coil can be controlled by the stomach itself. If the coils are biased to be straight, the stomach will bend the coil until all interior peaks are in contact. In this state, further compression of the coil must result in deformation of the peaks. Stiffness of this material can be controlled to allow significant or minimal deformations. Note, however, that all peaks need not be in contact for deformations to occur. Stomach geometry can be permitted to control this.

The coil may be comprised of a single body, or comprised of multiple bodies that are connected by hinges, snaps, wires, etc.

The current embodiment may also be made either partially or completely of bioabsorbable materials. Controlled degradation may be designed into the coil so that coil stiffness is altered over time. Additionally, the coil may be designed to break into numerous small pieces that will naturally pass without discomfort. Bioabsorbable sections may also be designed to release medications to the patient in a controlled way.

FIG. 18 shows a circular cross-section from the embodiment shown in FIG. 17.

FIG. 19 shows an elliptical cross-section from the embodiment shown in FIG. 17.

FIG. 20 shows a section of coil in an undeployed configuration. The coil may have some or all of the characteristics of the embodiment described in FIG. 17. A primary difference displayed in this figure is that the interior side of the coil will also have peaks and valleys that are designed to come in contact with adjacent pieces when deployed and are not all of the same shape. In FIG. 17 the interior peaks 131 and valleys 132 are uniform in shape and size. The peaks 133 and valleys 134 may also vary in shape and size along the length of the coil. The size and shape of the peaks 133 and valleys 134 control the deformed shape of the coil. Wider peaks and narrow valleys 135 will allow less bending whereas narrow peaks and wide valleys 136 will allow greater amounts of bending. Changes in geometry (angle, radius, depth, width, height, etc) allow excellent control over deformed coil shapes. For instance, a coil with uniform peaks 131 and valleys 132 will likely deform into a coil of constant radius, whereas non-uniform peaks 133 and valleys 134 can be arranged to create the U-shaped embodiment in FIG. 1. Cross sectional areas of the coil may be round (FIG. 18), elliptical (FIG. 19), or of other smooth shapes. The coil may contain holes 8 and 9 for stiffening elements (not shown).

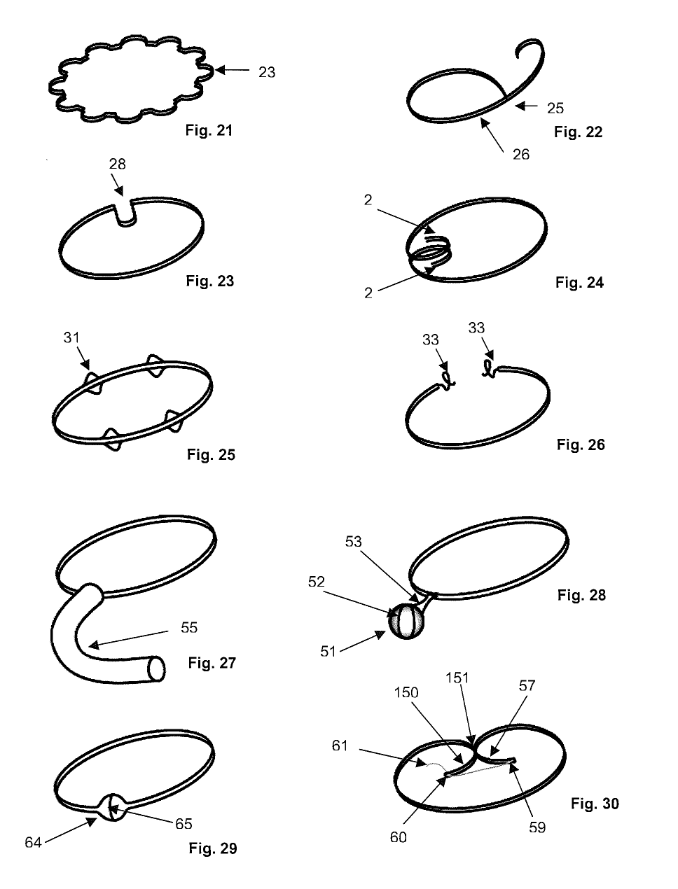

FIG. 21 shows an additional embodiment of the current invention. This figure shows a multitude of stress relieving loops 23 that allow the device to flex more easily when acted upon by severe stomach contractions. These loops may also serve similar functions as the peaks 3 and valleys 4 in FIG. 1.

FIG. 22 shows an additional embodiment of the current invention. The device in this figure has one end 25 attached to the main body 26 (Section 1 in FIG. 1) of the device. By adjusting the location of the attachment, the shape of the device is altered and the effect on the stomach modified. The attachment location can be directly or remotely repositioned in a variety of ways including ratchets, worn gears, servomotors, etc.

FIG. 23 shows an additional embodiment of the current invention. The device in this figure has a single stress relieving loop 28 similar to Device 22 added to the device to allow for flexing of the device when being acted upon by contractions of the stomach without having exposed ends as illustrated on the first U-shaped embodiment in FIG. 1.

FIG. 24 shows an additional embodiment of the current invention. This figure shows how the first U-shaped embodiment in FIG. 1 could have overlapping anti-migration sections 2 to further prevent migration and tissue damage. These overlapping regions maintain a smooth overall exterior shape when compressed and can be designed to maintain a relatively constant extension force on the stomach wall as the stomach changes size. An embodiment of this could take advantage of the constant stress region in the stress vs. strain diagram for nitinol materials.

FIG. 25 shows an additional embodiment of the current invention. The device in this figure has one or more out of plane elements 31 rising from the device to further extend and stimulate the stomach tissue. These elements include but are not limited to expanding super elastic materials such as nitinol, shape memory materials such as nitinol, a shape memory polymer, or expandable balloons.

FIG. 26 shows an additional embodiment of the current invention. The device in this figure has two ends terminated with out-of-plane coils 33 to prevent migration and erosion. Other out-of-plane features created from wires, balloons, or uncoiled sheets can serve the same purpose.

FIG. 27 shows an additional embodiment of the current invention. The device in this figure has a hollow sleeve 55 attached. The sleeve 55 is designed to migrate down the duodenum and provide a barrier to nutrient absorption. The sleeve 55 may be used to locate or fix the device within the stomach, the shape of the coil may be used to locate the device within the stomach, or the device may be fixed in place with suture, adhesive, anchors, etc.

FIG. 28 shows an additional embodiment of the current invention. The device in this figure has a pyloric plug 51 attached by element 53. This plug 51 is designed to sit down in the lower antrum, block flow of food through the pylorus. A plurality of grooves 52 allow for a controlled amount of food passage, resulting in delayed gastric emptying. The plug 51 may be used to locate or fix the device within the stomach, the shape of the coil may be used to locate the device within the stomach, or the device may be fixed in place with suture, adhesive, anchors, etc.

FIG. 29 shows an additional embodiment of the current invention. The device in this figure has a thickened section 64 that is also designed to partially block gastric emptying. Groove(s) 65 allows for a controlled amount of food passage in order to prevent complete obstruction. The thickened section 64 may be used to locate or fix the device within the stomach, the shape of the coil may be used to locate the device within the stomach, or the device may be fixed in place with suture, adhesive, anchors, etc.

FIG. 30 shows an additional embodiment of the current invention. This figure shows an alternative means of adjustment. Wire 61 is rigidly attached to device end coil 57 at point 59. Wire 61 is adjustably attached to device end coil 150 at point 60. When wire 61 is pulled, the two ends 60 and 59 are drawn together. The device could be configured so that end coils 57 and 150 bypass each other, resulting in a smaller coil as wire 61 is pulled. Alternatively, the device could be configures so that end coil 57 and 150 engage at point 151, resulting in a larger coil as the wire 61 is pulled.

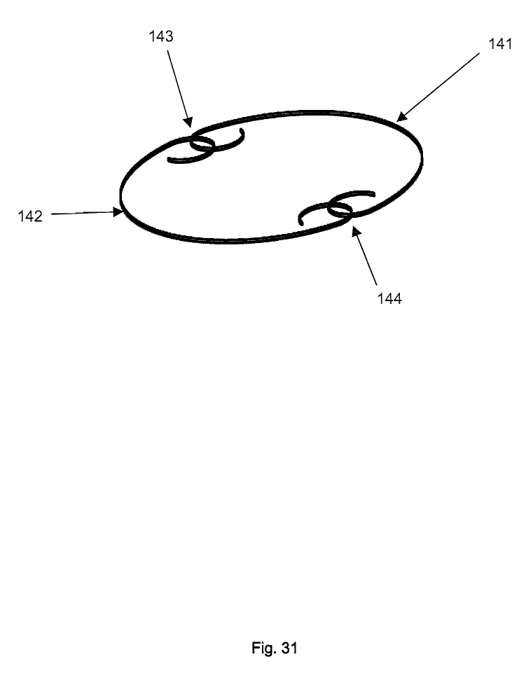

FIG. 31 illustrates how two individual coils 141 and 142, could be interconnected at points 143 and 144.

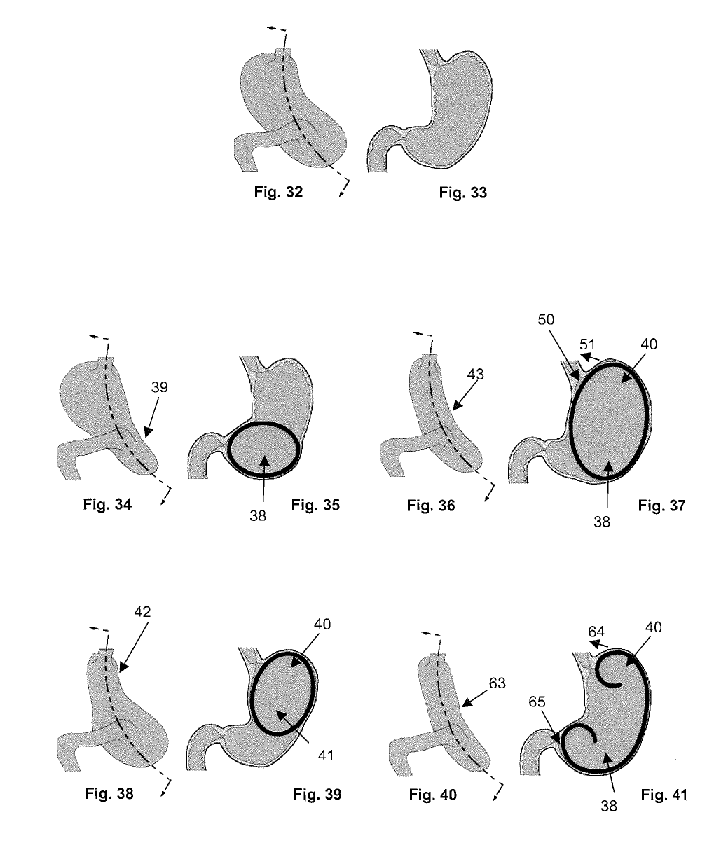

FIG. 32 shows the exterior shape of the stomach in an unaltered state prior to device insertion.

FIG. 33 shows the cross sectional view of the gastric lumen in an unaltered state prior to device insertion defined by FIG. 32.

FIG. 34 shows the exterior shape of the stomach in its altered state due to the deployment of the device shown in FIG. 35. Note the flattening 39 of the stomach in the area of the device compared to FIG. 32.

FIG. 35 shows the cross sectional view of the gastric lumen in its altered state due to the deployment of the closed loop device installed in the antrum 38 area of the stomach. Coils may be designed to more frequently sit in the indicated regions due to their size and/or shape. Devices can be encouraged to move about or rotate within these regions because of the overall shape or due to exterior features such as the peaks 3 and valleys 4 in FIG. 2. Alternatively, devices may be fixed in place with sutures, adhesives, or other external features (not shown).

FIG. 36 shows the exterior shape of the stomach in its altered state due to the deployment of the device shown in FIG. 37. Note the flattening 43 of the stomach in the area of the device compared to FIG. 32.

FIG. 37 shows the cross sectional view of the gastric lumen in its altered state due to the deployment of the closed loop device extending from the fundus 40 distally to the antrum 38 areas of the stomach. This illustration also shows how the device can create a restrictive inlet 50 that causes a bolus of food to be slowed as it enters the gastric lumen forcing the patient to eat more slowly and chew more thoroughly. The device could also be designed to provide a lateral force 51 at the fundus of the stomach, applying a closing force to the esophageal gastric (EG) junction, preventing GER and forcing the patient to eat more slowly and chew more thoroughly. Coils may be designed to more frequently sit in the indicated regions due to their size and/or shape. Devices can be encouraged to move about or rotate within these regions because of the overall shape or due to exterior features such as the peaks 3 and valleys 4 in FIG. 2. Alternatively, devices may be fixed in place with sutures, adhesives, or other external features (not shown).

FIG. 38 shows the exterior shape of the stomach in its altered state due to the deployment of the device shown in FIG. 39. Note the flattening 42 of the stomach in the area of the device compared to FIG. 32.

FIG. 39 shows the cross sectional view of the gastric lumen in its altered state due to the deployment of the closed loop device installed primarily in the fundus 40 and body 41 of the stomach. Coils may be designed to more frequently sit in the indicated regions due to their size and/or shape. Devices can be encouraged to move about or rotate within these regions because of the overall shape or due to exterior features such as the peaks 3 and valleys 4 in FIG. 2. Alternatively, devices may be fixed in place with sutures, adhesives, or other external features (not shown).

FIG. 40 shows the exterior shape of the stomach in its altered state due to the deployment of the device shown in FIG. 35. Note the flattening 63 of the stomach in the area of the device compared to FIG. 32.

FIG. 41 shows the cross sectional view of the gastric lumen in its altered state due to the deployment of an open looped device that extends from the fundus 40 to the antrum 38. Again a lateral pressure 64 can be applied to the EG junction. Additionally, this illustration shows how the device could apply a restriction outlet near the pylorus 65. Coils may be designed to more frequently sit in the indicated regions due to their size and/or shape. Devices can be encouraged to move about or rotate within these regions because of the overall shape or due to exterior features such as the peaks 3 and valleys 4 in FIG. 2. Alternatively, devices may be fixed in place with sutures, adhesives, or other external features (not shown).

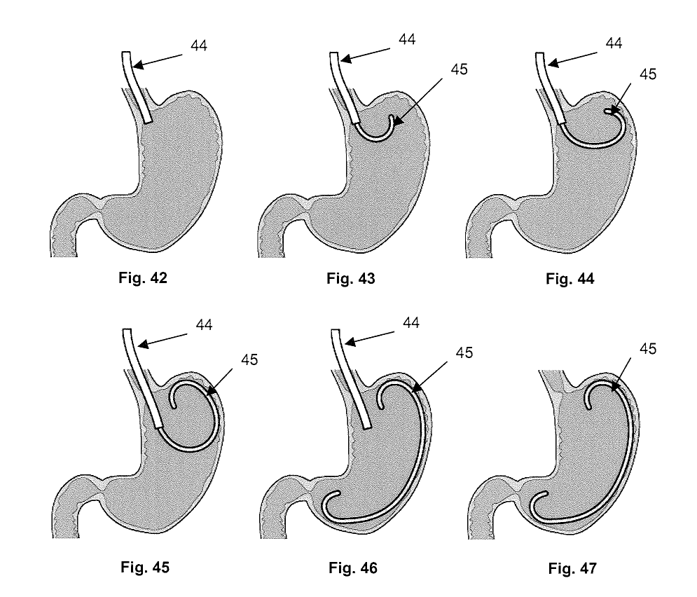

FIG. 42 shows one means of installing the open looped (U-shaped) embodiment of the device from FIG. 1. In this embodiment, an insertion device 44 is intubated into the stomach. A lumen therein contains the device 45 (FIG. 43) or alternatively, the device 45 is passed through the insertion device 44.

FIG. 43 shows one means of installing the open looped (U-shaped) embodiment of the device from FIG. 1. This illustration shows the device being pushed from the lumen of the insertion device 44 blindly, under direct or indirect (e.g. fluoroscopic) visualization. The device 45 preferentially bends as it is relieved from the insertion device.

FIG. 44 shows one means of installing the open looped (U-shaped) embodiment of the device from FIG. 1. This illustration is a continuation of FIG. 43 and shows the device being further pushed from the lumen of the insertion device 44 blindly, under direct or indirect (e.g. fluoroscopic) visualization. The device 45 preferentially bends as it is relieved from the insertion device.

FIG. 45 shows one means of installing the open looped (U-shaped) embodiment of the device from FIG. 1. This illustration is a continuation of FIG. 44 and shows the device being further pushed from the lumen of the insertion device 44 blindly, under direct or indirect (e.g. fluoroscopic) visualization. The device 45 preferentially bends as it is relieved from the insertion device.

FIG. 46 shows one means of installing the open looped (U-shaped) embodiment of the device from FIG. 1. This illustration shows the device 45 completely deployed and apart from the insertion device 44. The device 45 takes its final shape as it enters the gastric lumen. The final configuration of the device can be defined or driven by structures internal to the device such as the band 10 described in FIG. 2 or tensioning wires 61 described in FIG. 30. Alternatively, the final shape of the device can be driven by the tension in the stomach generated by the presence of the device. In this method, features on the device can limit the number of possible deployed configurations as is explained in the embodiment of FIGS. 17-20. Devices may also achieve their deformed shape through combinations of these mechanisms.

FIG. 47 shows the final step of installing the open looped (U-shaped) embodiment of the device from FIG. 1. This illustration shows the device 45 completely deployed and apart from the insertion device 44. Further, the insertion device 44 has been removed finishing this portion of the procedure.

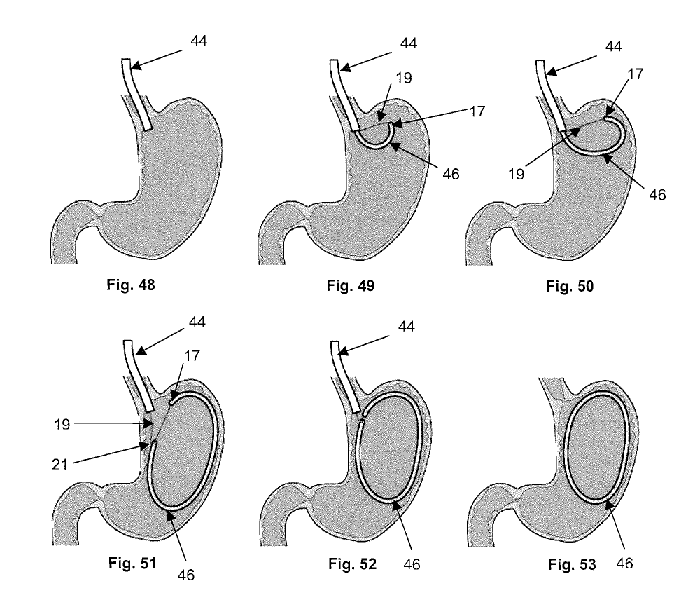

FIG. 48 shows a means of deploying and assembling the closed loop (circular or elliptical) configuration of the current invention. In this embodiment, an insertion device 44 is intubated into the stomach. A lumen therein contains the device 46 (FIG. 49) or alternatively, the device 46 is passed through the insertion device 44.

FIG. 49 shows a means of deploying and assembling the closed loop (circular or elliptical) configuration of the current invention. This illustration shows the device 46 being pushed from the lumen and into the stomach cavity while maintaining tension on cable 19, which is attached to the device 46 at point 17.

FIG. 50 shows a means of deploying and assembling the closed loop (circular or elliptical) configuration of the current invention. This illustration shows the device 46 being pushed further from the lumen and into the stomach cavity while maintaining tension on cable 19, which is attached to the device at point 17.

FIG. 51 shows a means of deploying and assembling the closed loop (circular or elliptical) configuration of the current invention. This illustration shows the device 46 being pushed completely from the lumen and into the stomach cavity while maintaining tension on cable 19, which is attached to the device at point 17. When the device 46 has exited the insertion device 44, the wire is pulled through hole 21.

FIG. 52 shows a means of deploying and assembling the closed loop (circular or elliptical) configuration of the current invention. This illustration shows tension being applied to cable 19 bringing both ends of the device 46 together closing the device to its final configuration inside the gastric lumen.

FIG. 53 shows a means of deploying and assembling the closed loop (circular or elliptical) configuration of the current invention. This illustration shows the device 46 in its final configuration. The final configuration of the device can be defined or driven by structures internal to the device such as the band 10 described in FIG. 2 or by the tension in the stomach generated by the presence of the device. Features on the device can be used to limit the number of possible deployed configurations as is explained in the embodiment of FIGS. 17-20. Devices may also achieve their deformed shape through combinations of these mechanisms.

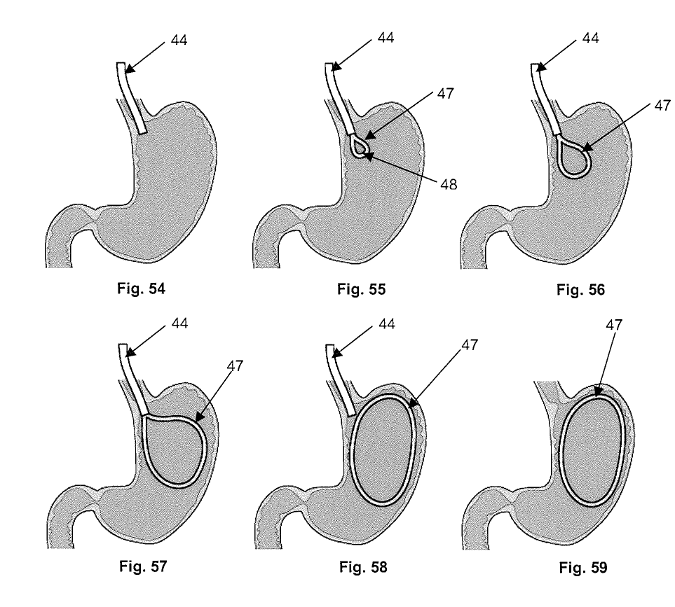

FIG. 54 shows an alternative means of deploying a closed loop embodiment of the current invention. In this embodiment, an insertion device 44 is intubated into the stomach. A lumen therein contains the device 47 (FIG. 55) or alternatively, the device is passed through the insertion device 44.

FIG. 55 shows the next step for deploying a closed loop embodiment of the current invention. This illustration shows the device 47 as it starts to exit the insertion device 44. The device 47 is folded over onto itself at point 48.

FIG. 56 shows the next step for deploying a closed loop embodiment of the current invention. This illustration shows the device 47 a bit further out of the insertion device 44. In this embodiment, point 48 is able to spring open once relieved from the insertion device 44.

FIG. 57 shows the next step for deploying a closed loop embodiment of the current invention. This illustration shows the device 47 a bit further out of the insertion device 44. In this embodiment it can now be seen that the device is forming into its final expanded configuration.

FIG. 58 shows the next step for deploying a closed loop embodiment of the current invention. This illustration shows the device 47 completely out of the insertion device 44 and in its final form, expanded and in contact with the stomach wall.

FIG. 59 shows the final step for deploying a closed loop embodiment of the current invention. This illustration shows the device 47 in its final form positioned within the stomach. The insertion device 44 has been removed finishing this portion of the procedure.

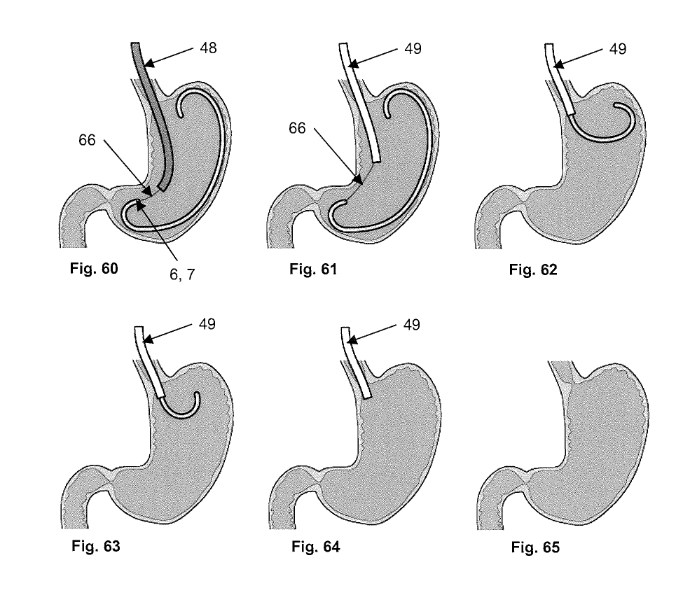

FIG. 60 shows a means of removing the open loop, U-shaped embodiment of FIG. 1. In this illustration, a pull wire 66 is attached to hole 6 or 7 by use of an endoscope 48.

FIG. 61 shows the next steps for removing the open loop, U-shaped embodiment of FIG. 1. In this illustration, the endoscope is removed and the wire 66 is left behind, extending out the mouth. The removal device 49 is intubated into the gastric lumen over top of the pull wire 66 with the pull wire running through a lumen within the removal device 49.

FIG. 62 shows the next step for removing the open loop, U-shaped embodiment of FIG. 1. In this illustration, the pull wire 66 is pulled, drawing the device into the lumen of the removal device 49.

FIG. 63 is a continuation of FIG. 62 showing the device further drawn into the lumen of the removal device 49.

FIG. 64 shows the next step for removing the open loop, U-shaped embodiment of FIG. 1. Once completely inside the removal device 49, the removal device is removed leaving the stomach as shown in FIG. 65.

FIG. 65 shows the stomach at the end of the procedure as described. However, in other embodiments, the removal device may be left in place for subsequent intervention. In this embodiment, the device is pulled completely out of the patient through the removal device 49.

FIG. 66 shows an open looped design similar to the design previously described in FIG. 1.

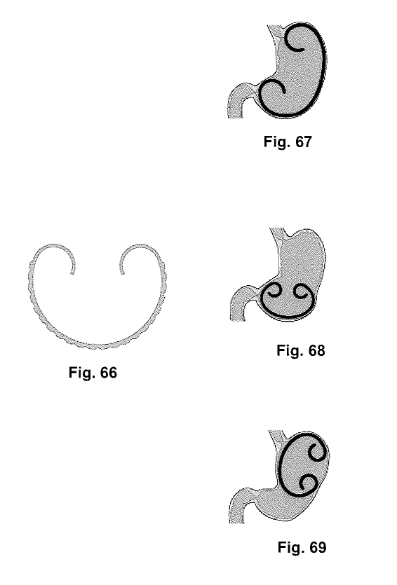

FIG. 67 further highlights how the open looped embodiment of the design previously described in FIG. 1 could be positioned in the stomach. It should be noted that the device is free to move within the stomach lumen and therefore it could reside in any number of planes within the stomach, not just the one illustrated.

FIG. 68 further highlights how the open looped embodiment of the design previously described in FIG. 1 could be positioned in the stomach. It should be noted that the device is free to move within the stomach lumen and therefore it could reside in any number of planes within the stomach, not just the one illustrated.

FIG. 69 further highlights how the open looped embodiment of the design previously described in FIG. 1 could be positioned in the stomach. It should be noted that the device is free to move within the stomach lumen and therefore it could reside in any number of planes within the stomach, not just the one illustrated.

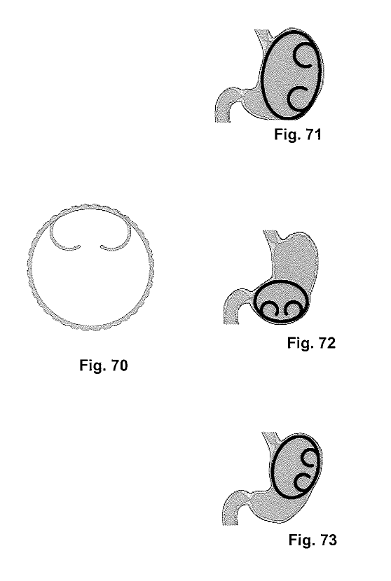

FIG. 70 shows an overlapping open looped design similar to the design previously described in FIG. 24.

FIG. 71 further highlights how the overlapping open looped design previously described in FIG. 24 could be positioned in the stomach. It should be noted that the device is free to move within the stomach lumen and therefore it could reside in any number of planes within the stomach, not just the one illustrated.

FIG. 72 further highlights how the overlapping open looped design previously described in FIG. 24 could be positioned in the stomach. It should be noted that the device is free to move within the stomach lumen and therefore it could reside in any number of planes within the stomach, not just the one illustrated.

FIG. 73 further highlights how the overlapping open looped design previously described in FIG. 24 could be positioned in the stomach. It should be noted that the device is free to move within the stomach lumen and therefore it could reside in any number of planes within the stomach, not just the one illustrated.

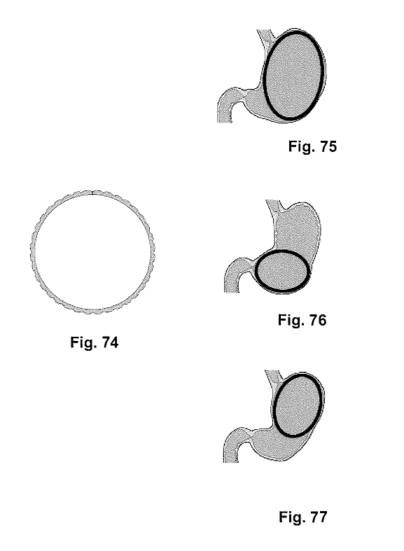

FIG. 74 shows a closed loop embodiment of the design similar to the design previously described in FIG. 6.

FIG. 75 further highlights how the closed loop embodiment of the design previously described in FIG. 6 could be positioned in the stomach. It should be noted that the device is free to move within the stomach lumen and therefore it could reside in any number of planes within the stomach, not just the one illustrated.

FIG. 76 further highlights how the closed loop embodiment of the design previously described in FIG. 6 could be positioned in the stomach. It should be noted that the device is free to move within the stomach lumen and therefore it could reside in any number of planes within the stomach, not just the one illustrated.

FIG. 77 further highlights how the closed loop embodiment of the design previously described in FIG. 6 could be positioned in the stomach. It should be noted that the device is free to move within the stomach lumen and therefore it could reside in any number of planes within the stomach, not just the one illustrated.

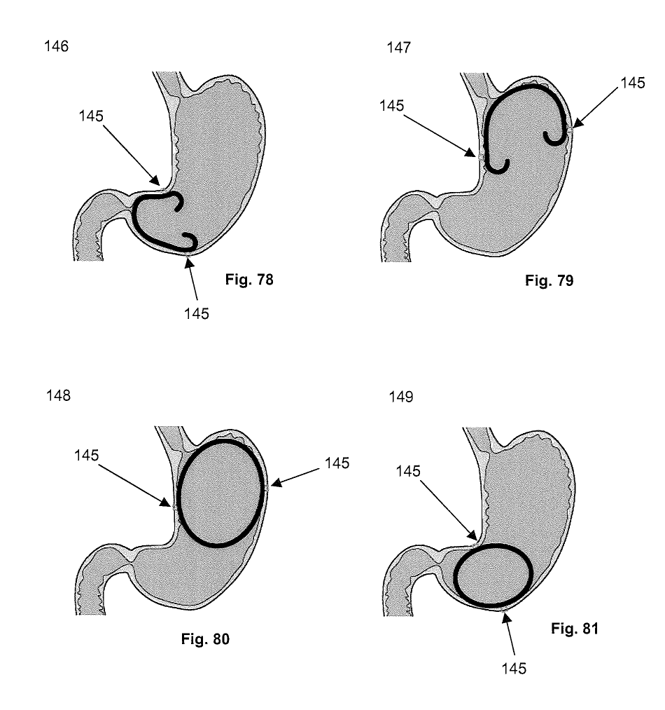

FIG. 78 illustrates how the device could be fixated to the stomach if it was desired to prevent migration, affect the shape or stiffness of the device or to ensure the device consistently acted upon a specific portion of the stomach wall. The figure shows the open looped design fixated in the antrum of the stomach where is could act to partially block the pylorus and/or activate a hormonal/neurological sense of satiety.

FIG. 79 illustrates how the device could be fixated to the stomach if it was desired to prevent migration, affect the shape or stiffness of the device or to ensure the device consistently acted upon a specific portion of the stomach wall. The figure shows the open looped embodiment fixated to the fundus and body of the stomach where it could act to partially block food intake at the EG Junction and/or stimulate stretch receptors to send an early sense of satiety.

FIG. 80 illustrates how the device could be fixated to the stomach if it was desired to prevent migration, affect the shape or stiffness of the device or to ensure the device consistently acted upon a specific portion of the stomach wall. The figure shows the closed loop embodiment fixated to the fundus and body of the stomach where it could act to partially block food intake at the EG Junction and/or stimulate stretch receptors to send an early sense of satiety.

FIG. 81 illustrates how the device could be fixated to the stomach if it was desired to prevent migration, affect the shape or stiffness of the device or to ensure the device consistently acted upon a specific portion of the stomach wall. The figure shows the closed loop design fixated in the antrum of the stomach where is could act to partially block the pylorus and/or activate a hormonal/neurological sense of satiety. Attachment mechanism 145 would be used to securely hold the devices in place. This attachment mechanism could include suture, t-fasteners, barbs, anchors, adhesives or any number of other tissue fastening techniques described in the art.

What is claimed:

1. An implant for placement within a hollow body organ, said implant comprising:
a. a member having an undeployed shape for delivery to the hollow body and a deployed shape for implantation therein;
b. said member having sufficient rigidity in its deployed shape to exert an outward force against an interior of the hollow body so as to bring together two substantially opposing surfaces of said hollow body.

2. The implant of claim 1 wherein said member is deformable having a relaxed configuration when in its deployed shape and a constrained configuration when in its undeployed shape.

3. The implant of claim 2 wherein said member is elastically deformable.

4. The implant of claim 1 wherein said member is deformable having a constrained configuration when in its deployed shape and a relaxed configuration when in its undeployed shape.

5. The implant of claim 4 wherein said member is elastically deformable.

6. The implant of claim 1 wherein said member comprised a plurality of segments movably connected to each other in said undeployed shape, and a means for attaching said segments to each other when placing said member in said deployed shape.

7. The implant of claim 1 wherein said member comprises a superelastic material.

8. The implant of claim 1 wherein said member has an exterior surface for contacting said hollow body and wherein said exterior surface has a plurality of peaks and valleys disposed thereon.

9. The implant of claim 1 wherein said member has an exterior surface for contacting said hollow body and wherein said member has an elastomer coating.

10. The implant of claim 1 further including a means for adjusting at least one of said:
deployed shape, rigidity or outward force.

11. The implant of claim 1 wherein said rigid member is substantially U-shaped in its deployed position.

12. The implant of claim 1 wherein said rigid member is substantially O-shaped in its deployed position.

13. An implant for placement within a hollow body, said implant comprising:
a. a member having an undeployed shape for delivery to the hollow body and a deployed shape for implantation therein;
b. said member having sufficient rigidity in its deployed shape to exert an outward force against an interior of the hollow body so as to bring together two substantially opposing surfaces of said hollow body;
c. said deployed shape of said member is sized such that said member is unable exit the hollow body through a natural opening therein.

14. The implant of claim 13 wherein said member is elastically deformable having a relaxed configuration when in its deployed shape and a constrained configuration when in its undeployed shape.

15. The implant of claim 13 wherein said member is elastically deformable having a constrained configuration when in its deployed shape and a relaxed configuration when in its undeployed shape.

16. The implant of claim 13 wherein said member comprised a plurality of rigid segments movably connected to each other in said undeployed shape, and a means for rigidly attaching said rings to each other when placing said member in said deployed shape.

17. The implant of claim 13 wherein said member comprises a superelastic material.

18. The implant of claim 13 wherein said member has an exterior surface for contacting said hollow body and wherein said exterior surface has a plurality of peaks and valleys disposed thereon.

19. The implant of claim 13 wherein said member has an exterior surface for contacting said hollow body and wherein said member has an elastomer coating.

20. The implant of claim 13 further including a means for adjusting at least one of said:
deployed shape, rigidity or outward force.

21. The implant of claim 13 wherein said rigid member is substantially U-shaped in its deployed position.

22. The implant of claim 13 wherein said rigid member is substantially O-shaped in its deployed position.

23. An method for inducing weight loss with a patient, said method comprising the steps of:
a. providing an implant for placement within the stomach, said implant comprising a member having an undeployed shape for delivery to the stomach and a deployed shape for implantation therein;
b. delivering said member to the stomach while in its undeployed shape;
c. exerting an outward force against an interior of the stomach so as to bring together two substantially opposing surfaces of said hollow body by placing said member in its deployed position.

24. The method of claim 23 wherein said step of exerting said outward force further includes the step of reducing the interior volume of the stomach.

25. The method of claim 23 wherein said implant is deployed through a natural orifice.

26. The method of claim 23 wherein said implant is deployed laparoscopically.

27. The method of claim 23 wherein said implant is deployed by open surgery techniques.

28. An method for inducing weight loss with a patient, said method comprising the steps of:
a. providing an implant for placement within the stomach, said implant comprising a member having an undeployed shape for delivery to the stomach and a deployed shape for implantation therein;
b. delivering said member to the stomach while in its undeployed shape;
c. exerting an outward force against an interior of the stomach so as to bring together two substantially opposing surfaces of said hollow body by placing said member in its deployed position; and
d. removing said implant from the stomach.

29. The method of claim 26 wherein said step of exerting said outward force further includes the step of reducing the interior volume of the stomach.

30. The method of claim 26 wherein said implant is deployed through a natural orifice.

31. The method of claim 26 wherein said implant is deployed laparoscopically.

32. The method of claim 26 wherein said implant is deployed by open surgery techniques.

33. The method of claim 26 wherein said implant is removed through a natural orifice.

34. The method of claim 26 wherein said implant is removed laparoscopically.

35. The method of claim 26 wherein said implant is removed by open surgery techniques.
